(19) United States
(12) Patent Application Publication
(10) Pub. No.: US 2009/0306229 A1
(43) Pub. Date: Dec. 10, 2009

(54) Perfum Composition for Expressing the Fragrance of Green Tea Flower

(76) Inventors: Ji Young Choi, Gyeonggi-do (KR); Byeong Bae Jeon, Gyeonggi-do (KR); Hyung Jye Seo, Gyeonggi-do (KR)

Correspondence Address: NIXON & VANDERHYE, PC, 901 NORTH GLEBE ROAD, 11TH FLOOR, ARLINGTON, VA, 22203, US

(21) Appl. No.: 12/084,466

(22) PCT Filed: Nov. 2, 2006
(86) PCT No.: PCT/KR2006/004532

§ 371 Date: Feb. 24, 2009

(30) Foreign Application Priority Data
Nov. 10, 2005 (KR) 10-2005-0107434

(51) Int. Cl. A61K 8/33; A61K 8/34; A61K 8/35; A61K 8/37; A61K 8/49; A61Q 13/00

(52) U.S. Cl. 514/784; 512/25; 512/27; 512/20; 512/26; 512/21; 512/10; 514/772; 514/788

(57) ABSTRACT

Disclosed is a perfume composition having a fragrance like that of green tea flowers. The perfume composition contains artificial synthetic substance trans-4,8-dimethyl-1,3,7-nonatriene or lilac alcohol, in addition to the main fragrant components of green tea flowers, analyzed by an SPME method or a solvent extraction method, and thus has a fragrance like that of the characteristic fragrance of green tea flowers and, at the same time, has high preference.

### TECHNICAL FIELD

The present invention relates to a perfume composition having a fragrance like that of green tea flowers, and more particularly to a perfume composition, which contains artificial synthetic substance trans-4,8-dimethyl-1,3,7-nonatriene or lilac alcohol, in addition to the main fragrant components of green tea flowers, analyzed by an SPME method or a solvent extraction method, and thus has a fragrance like the characteristic fragrance of green tea flowers and, at the same time, has high preference.

### BACKGROUND ART

Tea trees are evergreen shrubs growing in, for example, the southern part of Korea, and the leaves thereof are leathery, are long oval in shape, have fine saw-tooth edges, and are 4-10 cm in length and 2-3 cm in width. The flowers thereof are white flowers having a size of about 2-2.5 cm, and a group of 1-3 flower blossoms is attached to the leaf axils and droops downward. Also, the flowers bloom during a period from the middle of October to December.

Particularly, the leaves of green tea trees (Camellia sinensis var. sinenis cv. Shuixian and Maoxie) have been widely used for a long time as important favorite beverages in Korea, China, Japan and the like. Because the quality of green tea changes depending on the fragrant components of the tea leaves, many studies on the fragrant components of the green tea leaves have been already conducted.

On the other hand, the flowers of green tea trees (Camellia sinensis var. sinenis cv. Shuixian and Maoxie) are not known with respect to their effective value, are not helpful in the production of the green tea leaves, and thus are almost wasted. However, the green tea flower has characteristic fresh fragrance, and thus receives attention as a fragrant plant.

To prepare products having a fragrance like that of such green tea flowers, various methods are conventionally used. Examples of these conventional methods include a solvent extraction method of making absolute oils using the fragrant components of green tea flowers, and a steam distillation method of making essential oils using the fragrant components. Recently, a head space method and a solid-phase micro-extraction (SPME; SUPELCO international, Vol. 13, No. 4, pp. 9-10) have been developed which collect and analyze the fragrant components in a rapid and convenient manner without pretreatment of samples.

### DISCLOSURE

### Technical Problem

Accordingly, the present inventors have made efforts to develop a product having a fragrance like that of green tea flowers, using the SPME or solvent extraction method effective in analyzing the fragrant components of living natural materials. As a result, the present inventors have found that acetophenon, phenylethyl alcohol and the like are the main fragrant components of green tea flowers, and have attempted to develop a product having a fragrance like that of green tea flowers, using these components. However, the results of sensory tests conducted by the present inventors showed that the fragrance of green tea flowers could not be produced only with these components.

Accordingly, the present inventors have conducted studies to develop a product having a fragrance like that of green tea flowers and, as a result, found that a perfume composition, which contains, in addition to the main fragrant components of green tea flowers, artificial synthetic fragrant substance E-4,8-dimethyl-1,3,7-nonatriene or lilac alcohol, as active ingredients, can emit a fragrance like the characteristic fragrance of green tea flowers and has high preference, thereby completing the present invention.

Therefore, it is an object of the present invention to provide a perfume composition, which can emit a fragrance like the characteristic fragrance of green tea flowers and has high preference, and a preparation method thereof.

Another object of the present invention is to provide a cosmetic composition containing said perfume composition as a constituent component.

### Technical Solution

To achieve the above objects, in one aspect, the present invention provides a method for preparing a perfume composition having a fragrance like that of green tea flowers, the method comprising: analyzing the fragrant components of green tea flowers using an SPME method or a solvent extraction method; subjecting each of the fragrant components to sensory evaluation to select the main fragrant components of the green tea flowers; and adding a fragrance-enhancing perfume to the main fragrant components.

In another aspect, the present invention provides a perfume composition containing: main fragrant components of green tea flowers, analyzed using an SPME method or a solvent extraction method; and artificial synthetic substance E-4,8-dimethyl-1,3,7-nonatriene or lilac alcohol.

Hereinafter, the present invention will be described in further detail.

In the present invention, an SPME method or a solvent extraction method is used as a method for analyzing the fragrant components of green tea flowers. In the SPME method, fragrant components adsorbed to a fiber material are desorbed at the inlet of a GC-MS column without any special pretreatment and are rapidly injected into the column. Thus, the SPME method greatly reduces analysis time and is advantageous for analyzing the fragrant components of green tea flowers in a living state. Meanwhile, the solvent extraction method is a method comprising immersing green tea flowers in a solvent in order to find fragrant components, which are contained in small amounts, but have a great effect on fragrance, and extracting the fragrant components. The solvent extraction method is time-consuming compared to the SPME method, but is advantageous for finding various components contributing to making the fragrance of green tea flower fragrance.

The main fragrant components of green tea flowers, analyzed by the SPME method, include acetophenon, alpha-phenylethyl alcohol, beta-phenylethyl alcohol and linalool. The secondary fragrant components of green tea flowers, analyzed by the SPME method, include 2-pentanone, 2-pentanol, benzyl alcohol, Z-3-hexenyl acetate, geraniol, 1-penten-3-ol, acetoin, Z-3-hexenol, linalool oxide, Z-4-hexenyl butyrate, and limonene.

Meanwhile, the main fragrant components of green tea flowers, analyzed by the solvent extraction method, include acetophenon, alpha-methylbenzyl alcohol, and 2-phenylethyl alcohol. The secondary fragrant components of green tea flowers, analyzed by the solvent extraction method, include linalool, geraniol, linlool oxide, methyl salicylate, indole, and vanillin.

Each of the fragrant components analyzed as described above is subjected to a sensory test by a professional evaluation group consisting of flavorists to analyze the fragrance thereof. Among the analyzed fragrances, the main fragrant components of green tea flowers, which are highly similar to the fragrance of green tea flowers and have excellent preference, are selected, and a fragrance-enhancing perfume is then added to the main fragrant components, thus preparing a perfume composition having a fragrance like that of green tea flowers.

In the present invention, when the SPME method is used to analyze the fragrant components of green tea flowers, the following components are analyzed as the main fragrant components of green tea flowers: acetophenon, alpha-phenylethyl alcohol, beta-phenylethyl alcohol, and linalool. Also, the following components are analyzed as the secondary fragrant components: 2-pentanone, 2-pentanol, benzyl alcohol, Z-3-hexenyl acetate, geraniol, 1-penten-3-ol, acetoin, Z-3-hexenol, linalool oxide, Z-4-hexenyl butyrate, and limonene. To these analyzed fragrant components, artificial synthetic substance E-4,8-dimethyl-1,3,7-nonatriene having strong floral neroli fragrance is added as an active ingredient, thus providing a perfume composition, which can emit a fragrance like the characteristic fragrance of green tea flowers and shows high preference.

The perfume composition preferably contains, based on 100 parts by weight of the composition, 44.00-51.00 wt % of acetophenon, 8.20-13.00 wt % of alpha-phenylethyl alcohol, 3.50-6.20 wt % of beta-phenylethyl alcohol, 1.05-3.50 wt % of linalool, and 1-10 wt % of E-4,8-dimethyl-1,3,7-nonatriene as an artificial synthetic substance.

In addition, the perfume composition may further contain, as secondary fragrant components, 6.00-9.97 wt % of pentanone, 4.20-7.23 wt % of pentanol, 2.75-4.95 wt % of benzyl alcohol, 2.00-3.75 wt % of Z-3-hexenyl acetate, 1.76-3.57 wt % of geraniol, 2.36-2.40 of 1-penten-3-ol, 1.21-2.83 wt % of acetoin, 0.64-1.05 wt % of Z-3-hexenol, 0.23-0.78 wt % of linalool oxide, 0.02-0.10 wt % of Z-4-hexenyl butyrate, and 0.01-0.07 wt % of limonene.

If acetophenon, alpha-phenylethyl alcohol, beta-phenylethyl alcohol, linalool and artificial synthetic substance E-4,8-dimethyl-1,3,7-nonatriene, which are main components for producing a fragrance like that of green tea flowers, are used in amounts deviating from the above-specified ranges, the similarity of fragrance to the fragrance of green tea flowers will be decreased, and the preference of fragrance will also be decreased.

Although the other components are preferably used in the above-specified content ranges, such components can also be used in amounts deviating from the above ranges as long as they have no effect on the production of the fragrance of green tea flowers, because they have no great effect on the fragrance of green tea flowers.

In the present invention, when the solvent extraction method is used to analyze the fragrance of green tea flowers, the following components are analyzed as the main fragrant components of green tea flowers: acetophenon, alpha-methylbenzyl alcohol, and 2-phenylethyl alcohol. In addition, the following components are analyzed as the secondary fragrant components of green tea flowers: linalool, geraniol, linalool oxide, methyl salicylate, indole, and vanillin. To these fragrant components, artificial synthetic substance lilac alcohol is added as an active ingredient, thus preparing a perfume composition, which can emit a fragrance like the characteristic fragrance of green tea flowers and shows high preference.

The composition preferably contains, based on 100 parts by weight of the composition, 26.0-35.0 wt % of acetophenon, 20.0-27.0 wt % of alpha-methylbenzyl alcohol, 8.5-11.0 wt % of 2-phenylethyl alcohol, and 0.1-7.4 wt % of artificial synthetic substance lilac alcohol.

In addition, the composition may further contain 3.0-4.0 wt % of linalool, 1.5-2.5 wt % of geraniol, 2.0-3.0 wt % of linalool oxide, 0.1-1.0 wt % of methyl salicylate, 0.01-0.1 wt % of indole, and 0.01-0.1 wt % of vanillin.

If acetophenon, alpha-methylbenzyl alcohol, 2-phenylethyl alcohol and artificial synthetic substance lilac alcohol, which are main components for producing a fragrance like that of green tea flowers, are used in amounts deviating from the above-specified ranges, the similarity of fragrance to the fragrance of green tea flowers will be decreased, and the preference of fragrance will also be decreased.

Although the other components are preferably used in the above-specified content ranges, such components can also be used in amounts deviating from the above ranges as long as they have no effect on the production of the fragrance of green tea flowers, because they have no great effect on the fragrance of green tea flowers.

The composition according to the present invention, having the components and contents as described above, can be added to a cosmetic composition in an amount of 0.1-25.0 wt % based on the total weight of the cosmetic composition. For example, the composition can be formulated into not only cosmetics, such as lotion, cream and emulsion, but also preparations for external application, including perfumes, ointments, solubilized phases, suspensions, gels, sprays, pastas, plasters, patches, and liquid plasters. The content of the inventive composition in each of these formulations can be suitably selected depending on a conventional technique known in the art so as to achieve the desired effects, but the inventive composition is not limited only to these formulations and can also be added to any base known in the art.

In the present invention, perfume compositions were made based on the fragrant components of green tea flowers, analyzed by the SPME method or the solvent extraction method. These compositions were subjected to olfactory sensory tests to examine the similarity of fragrance thereof to the fragrance of green tea flowers and the preference of fragrance thereof. The sensory tests were conducted by professional flavorists and general persons, and the similarity of fragrance of the compositions to the fragrance of green tea flowers and the preference of fragrance thereof were evaluated through questionnaire survey.

### MODE FOR INVENTION

Hereinafter, the present invention will be described in further detail with reference to examples. It is to be understood, however, that these examples are illustrative only and the scope of the present invention is not limited thereto.

### Example 1

### Perfume Compositions Having Fragrance Like that of Green Tea Flowers, Based on SPME Method

1-1: Analysis of Fragrance of Green Tea Flowers Using SPME Method

Because the fragrance of green tea flowers is emitted after dew is completely removed therefrom, a bloom of flowers of Kanayamidori green tea trees, having strong fragrance, were maintained on a 85-μm polyacrylate fiber for 5 hours starting at 8 A.M. in order to collect fragrant components. The collection of fragrant components was conducted at a temperature of 18° C. in wind-free conditions in Pacific Seogwang Dawon, Seogwang-ri, Jeju-do, Korea.

After the fragrant components were collected, the fiber was sealed, put into the inlet of a GC-MS column, desorbed for 2 minutes, and subjected to GC-MS analysis. The GC-MS analysis was carried out in the following conditions.

Analysis Conditions

Analysis instrument: HP 5890 II GC

### Detector: HP 5972 MSD

Column: DB-1 (60 m×0.25 mm×0.25 um)

### Carrier gas: He

Inlet temperature: 250° C.
Detection temperature: 280° C.
Oven temperature: 70-220° C. (3° C./min)
Ion voltage: 70 eV
Desorption time: 2 min

As a result, the fragrant components of green tea flowers, analyzed by the SPME method, are shown in Table 1 below.

TABLE 1

| Name of fragrant components | Content (wt %) |
|---|---|
| Acetophenon | 50.1000 |
| 2-pentanone | 13.2000 |
| alpha-phenylethyl alcohol | 11.3000 |
| beta-phenylethyl alcohol | 5.1000 |
| 2-pentanol | 4.7000 |
| Benzyl alcohol | 3.5000 |
| Linalool | 2.3000 |
| cis-3-hexenyl acetate | 1.7000 |
| Geraniol | 1.6000 |
| 1-penten-3-ol | 1.5000 |
| Acetoin | 1.2000 |
| cis-3-hexenol | 0.8000 |
| Linalool oxide | 0.3000 |
| cis-4-hexenyl butyrate | 0.0400 |
| Limonene | 0.0300 |
| Others | 2.63 |
| Total content | 100.0000 |

As can be seen in Table 1, the green tea flowers contain, as main fragrant components, acetophenon, 2-pentanone, alpha-phenylethyl alcohol, beta- and alpha-phenylethyl alcohols and 2-pentanol, and the content of these main components in the total fragrant components is 84.4 wt %.

1-2. Comparative Sensory Evaluation of Fragrance Between Perfume Composition Based on Analysis Results and Natural Green Tea Flower

Based on the above analysis results, a perfume composition (sample B) shown in Table 2, and the similarity of fragrance between the perfume composition and natural green tea flowers was examined through sensory evaluation.

TABLE 2

| Name of fragrant components | Content (wt %) |
|---|---|
| acetophenon | 50.10000 |
| 2-pentanone | 13.2000 |
| alpha-phenylethyl alcohol | 11.3000 |
| beta-phenylethyl alcohol | 5.1000 |
| 2-pentanol | 4.7000 |
| Benzyl alcohol | 3.5000 |
| Linalool | 2.3000 |
| cis-3-hexenyl acetate | 1.7000 |
| Geraniol | 1.6000 |
| 1-penten-3-ol | 1.5000 |
| Acetoin | 1.2000 |
| cis-3-hexenol | 0.8000 |
| Linalool oxide | 0.3000 |
| cis-4-hexenyl butyrate | 0.0400 |
| Limonene | 0.0300 |
| DEP (solvent) | 2.63 |
| Total content | 100.0000 |

The sensory evaluation was conducted on twenty 20-45-year-old general men and women, and the similarity of fragrance (question 1) between the green tea flowers in full bloom (sample A) and the perfume composition (sample B) obtained using the SPME method, and the preference of fragrance (question 2), were examined by causing the evaluation panels to smelling the fragrance of each of the samples A and B and answering a questionnaire shown in Table 3 below. The evaluation results are shown in Table 4 below.

TABLE 3
Questionnaire

(1) Question 1: how the fragrance of the sample A is similar to the
fragrance of sample B?
1) completely dissimilar
2) not particularly similar
3) so-so
4) somewhat similar
5) significantly similar
(2) Question 2: how you like the fragrance of the sample B?
1) completely dislike
2) not particularly like
3) so-so
4) like
5) significantly like

TABLE 4

| Evaluation | Question 1 | Question 2 |
|---|---|---|
| panel No. | (similarity) | (preference) |
| 1 | 3 | 1 |
| 2 | 2 | 1 |
| 3 | 1 | 2 |
| 4 | 2 | 2 |
| 5 | 1 | 2 |
| 6 | 1 | 2 |
| 7 | 2 | 2 |
| 8 | 2 | 4 |
| 9 | 2 | 2 |
| 10 | 2 | 2 |
| 11 | 2 | 2 |
| 12 | 3 | 3 |
| 13 | 3 | 2 |
| 14 | 2 | 1 |
| 15 | 1 | 3 |
| 16 | 1 | 2 |
| 17 | 3 | 2 |
| 18 | 2 | 1 |
| 19 | 3 | 2 |
| 20 | 1 | 2 |
| Average | 1.95 | 2.00 |

As can be seen in Table 4, the perfume composition (sample B) had a fragrance greatly different from the fragrance of natural green tea flowers, and showed low preference.

1-3: Analysis of Fragrance and Comparison of Fragrance with Green Tea Flowers, Conducted by Professional Evaluation Group

The perfume composition consisting of the components analyzed by the SPME method had no similarity to the fragrance of green tea flowers. Thus, each of the fragrant components was subjected to sensory evaluation by a professional evaluation group consisting of flavorists. As a result, it was found that, among the fragrant components of green tea flowers, four components, including acetophenon making Neroli fragrance, alpha- and beta-phenylethyl alcohol making rose fragrance, and linalool making herbal floral fragrance, are main components making the characteristic fragrance of green tea flowers. Particularly, when green tea flowers were compared to other kinds of flowers with respect to the contents of the two phenylethyl alcohols, it was found that the green tea flowers contained alpha-phenylethyl alcohol in an amount larger than beta-phenylethyl alcohol and emitted green tea fragrance, which was faint and had good diffusion properties and long-lasting properties.

Meanwhile, it was confirmed through sensory evaluation that 2-pentanone and 2-pentanol, which were classified as the main fragrant components together with acetophenon, alpha-phenylethyl alcohol, beta-phenylethyl alcohol and linalool in the SPME analysis results, had no particular effect on making the fragrance of green tea flowers.

1-4: Preparation of New Perfume Compositions Having Varying Contents of Four Main Components

From the results of Example 1-3, it could be seen that the main components for the characteristic fragrance of green tea flowers were acetophenon, alpha- and beta-phenylethyl alcohols, and linalool. Thus, in order to prepare a perfume composition, which contains these four components to emit the fragrance of green tea flowers and, at the same time, has high preference, new perfume compositions #1-1 to #1-8 were prepared. The perfume compositions #1-1 to #1-8 were prepared such that the ratio between these four components was maintained constant while the contents of the four components in the compositions shown in Table 5 below were changed in the range of 60-95 wt %.

TABLE 5

| | Perfume composition (total content of main fragrant components) | | | | | | | |
|---|---|---|---|---|---|---|---|---|
| Name of fragrant | #1-1 | #1-2 | #1-3 | #1-4 | #1-5 | #1-6 | #1-7 | #1-8 |
| components | 60 wt % | 65 wt % | 70 wt % | 75 wt % | 80 wt % | 85 wt % | 90 wt % | 95 wt % |
| Acetophenon | 43.80 | 47.44 | 51.10 | 54.75 | 58.45 | 62.10 | 65.73 | 69.20 |
| alpha-phenylethyl | 9.79 | 10.62 | 11.45 | 12.25 | 13.00 | 13.75 | 14.75 | 15.54 |
| alcohol | | | | | | | | |
| Beta-phenylethyl | 4.41 | 4.78 | 5.15 | 5.53 | 5.90 | 6.30 | 6.50 | 7.07 |
| alcohol | | | | | | | | |
| Linalool | 2.00 | 2.16 | 2.30 | 2.47 | 2.65 | 2.85 | 3.02 | 3.19 |
| 2-pentanone | 10.67 | 9.31 | 7.98 | 6.65 | 5.30 | 3.95 | 2.65 | 1.32 |
| 2-pentanol | 7.94 | 6.93 | 5.93 | 4.90 | 3.95 | 2.96 | 1.96 | 0.98 |
| Benzyl alcohol | 5.20 | 4.56 | 3.90 | 3.25 | 2.60 | 1.96 | 1.30 | 0.65 |
| cis-3-hexenyl | 3.84 | 3.36 | 2.89 | 2.40 | 1.93 | 1.45 | 0.97 | 0.48 |
| acetate | | | | | | | | |
| Geraniol | 3.62 | 3.17 | 2.72 | 2.28 | 1.82 | 1.37 | 0.91 | 0.46 |
| 1-penten-3-ol | 3.40 | 2.96 | 2.55 | 2.14 | 1.70 | 1.28 | 0.86 | 0.43 |
| Acetoin | 2.72 | 2.37 | 2.05 | 1.71 | 1.37 | 1.03 | 0.68 | 0.34 |
| cis-3-hexenol | 1.82 | 1.60 | 1.36 | 1.14 | 0.91 | 0.68 | 0.46 | 0.23 |
| Linalool oxide | 0.66 | 0.60 | 0.50 | 0.43 | 0.34 | 0.26 | 0.17 | 0.09 |
| cis-4-hexenyl | 0.07 | 0.08 | 0.07 | 0.06 | 0.05 | 0.03 | 0.02 | 0.01 |
| butyrate | | | | | | | | |
| Limonene | 0.06 | 0.06 | 0.05 | 0.04 | 0.03 | 0.03 | 0.02 | 0.01 |
| Total content | 100.0 | 100.0 | 100.0 | 100.0 | 100.0 | 100.0 | 100.0 | 100.0 |

1-5. Sensory Evaluation of New Perfume Compositions

The above eight new perfume compositions were examined with respect to the similarity of fragrance to the fragrance of green tea flowers and the preference of fragrance using sensory evaluation in the same manner as described in Table 3 above. Meanwhile, after the comparative sensory evaluation of two perfume combinations was conducted, a breathing time of 5 minutes was given to eliminate olfactory paralysis. Sensory evaluation results for the similarity of fragrance between the new perfume compositions and green tea flowers, and the preference of fragrance, are shown in Table 6 below.

TABLE 6

| | Question 1 (similarity) | | | | | | | | Question 2 (preference) | | | | | | | |
|---|---|---|---|---|---|---|---|---|---|---|---|---|---|---|---|---|
| Evaluation | Perfume composition No. | | | | | | | | Perfume composition No. | | | | | | | |
| panels | #1-1 | #1-2 | #1-3 | #1-4 | #1-5 | #1-6 | #1-7 | #1-8 | #1-1 | #1-2 | #1-3 | #1-4 | #1-5 | #1-6 | #1-7 | #1-8 |
| 1 | 2.0 | 4.0 | 3.0 | 3.0 | 2.0 | 3.0 | 2.0 | 2.0 | 2.0 | 3.0 | 2.0 | 3.0 | 1.0 | 1.0 | 2.0 | 2.0 |
| 2 | 2.0 | 3.0 | 3.0 | 3.0 | 3.0 | 2.0 | 2.0 | 1.0 | 3.0 | 3.0 | 3.0 | 3.0 | 2.0 | 1.0 | 1.0 | 2.0 |
| 3 | 3.0 | 3.0 | 4.0 | 3.0 | 3.0 | 1.0 | 2.0 | 2.0 | 3.0 | 4.0 | 3.0 | 3.0 | 2.0 | 2.0 | 2.0 | 1.0 |
| 4 | 2.0 | 3.0 | 3.0 | 2.0 | 2.0 | 2.0 | 1.0 | 2.0 | 4.0 | 3.0 | 2.0 | 2.0 | 3.0 | 2.0 | 2.0 | 2.0 |
| 5 | 2.0 | 4.0 | 2.0 | 3.0 | 2.0 | 1.0 | 2.0 | 3.0 | 3.0 | 3.0 | 2.0 | 3.0 | 4.0 | 2.0 | 3.0 | 1.0 |
| 6 | 2.0 | 4.0 | 3.0 | 3.0 | 2.0 | 1.0 | 1.0 | 2.0 | 3.0 | 4.0 | 3.0 | 2.0 | 3.0 | 2.0 | 2.0 | 2.0 |
| 7 | 3.0 | 4.0 | 2.0 | 4.0 | 2.0 | 2.0 | 2.0 | 2.0 | 2.0 | 2.0 | 3.0 | 1.0 | 3.0 | 2.0 | 2.0 | 2.0 |
| 8 | 2.0 | 3.0 | 3.0 | 3.0 | 2.0 | 2.0 | 2.0 | 1.0 | 3.0 | 3.0 | 2.0 | 2.0 | 3.0 | 4.0 | 2.0 | 1.0 |
| 9 | 3.0 | 4.0 | 3.0 | 3.0 | 2.0 | 2.0 | 1.0 | 3.0 | 2.0 | 3.0 | 2.0 | 3.0 | 3.0 | 2.0 | 1.0 | 2.0 |
| 10 | 2.0 | 4.0 | 2.0 | 3.0 | 3.0 | 2.0 | 2.0 | 2.0 | 3.0 | 4.0 | 2.0 | 3.0 | 2.0 | 2.0 | 2.0 | 1.0 |
| 11 | 2.0 | 3.0 | 4.0 | 2.0 | 2.0 | 2.0 | 3.0 | 1.0 | 3.0 | 3.0 | 3.0 | 2.0 | 3.0 | 2.0 | 1.0 | 2.0 |
| 12 | 3.0 | 4.0 | 3.0 | 3.0 | 3.0 | 3.0 | 2.0 | 2.0 | 2.0 | 3.0 | 2.0 | 3.0 | 2.0 | 3.0 | 2.0 | 3.0 |
| 13 | 2.0 | 2.0 | 2.0 | 3.0 | 3.0 | 3.0 | 2.0 | 1.0 | 2.0 | 3.0 | 2.0 | 2.0 | 2.0 | 2.0 | 1.0 | 2.0 |
| 14 | 3.0 | 2.0 | 3.0 | 2.0 | 3.0 | 2.0 | 1.0 | 2.0 | 3.0 | 2.0 | 3.0 | 3.0 | 2.0 | 1.0 | 2.0 | 3.0 |
| 15 | 3.0 | 4.0 | 3.0 | 3.0 | 2.0 | 1.0 | 1.0 | 2.0 | 2.0 | 3.0 | 2.0 | 3.0 | 2.0 | 3.0 | 2.0 | 3.0 |
| 16 | 2.0 | 3.0 | 3.0 | 2.0 | 2.0 | 1.0 | 1.0 | 1.0 | 3.0 | 3.0 | 3.0 | 3.0 | 2.0 | 2.0 | 1.0 | 2.0 |
| 17 | 2.0 | 3.0 | 3.0 | 3.0 | 2.0 | 3.0 | 2.0 | 1.0 | 4.0 | 3.0 | 3.0 | 3.0 | 3.0 | 2.0 | 2.0 | 3.0 |
| 18 | 1.0 | 4.0 | 3.0 | 3.0 | 3.0 | 2.0 | 1.0 | 1.0 | 2.0 | 4.0 | 3.0 | 3.0 | 2.0 | 1.0 | 1.0 | 3.0 |
| 19 | 2.0 | 4.0 | 3.0 | 3.0 | 3.0 | 3.0 | 2.0 | 2.0 | 1.0 | 4.0 | 4.0 | 3.0 | 3.0 | 2.0 | 2.0 | 2.0 |
| 20 | 2.0 | 3.0 | 3.0 | 3.0 | 2.0 | 1.0 | 2.0 | 2.0 | 1.0 | 3.0 | 2.0 | 2.0 | 2.0 | 2.0 | 2.0 | 3.0 |
| Average | 2.25 | 3.4 | 2.90 | 2.85 | 2.40 | 1.95 | 1.70 | 1.75 | 2.55 | 3.15 | 2.55 | 2.60 | 2.45 | 2.00 | 1.75 | 2.10 |

As shown in Table 6, the sample #1-2, in which the content of the four main components was 65 wt %, showed the highest similarity and preference among the eight perfume compositions, but all the similarity and preference of the sample #1-2 were not as high as expected.

1-6: Preparation of Perfume Compositions Having Improved Fragrance Based on Analysis Conducted by Professional Evaluation Group

To improve the similarity and preference of the sample #1-2, which was shown to be most similar to the fragrance of green tea flowers in Table 6 above, new perfume compositions were prepared by adding E-4,8-dimethyl-1,3,7-nonatriene having strong floral nenoli fragrance to the sample #1-2 in which the total content of acetophenon, alpha-phenylethyl alcohol, beta-phenylethyl alcohol and linalool was 65%. The E-4,8-dimethyl-1,3,7-nonatriene was added in concentrations of 1%, 3%, 5%, 8%, 10%, 13% and 15%. These perfume compositions are shown in Table 7 below.

TABLE 7

| | Perfume compositions | | | | | | |
|---|---|---|---|---|---|---|---|
| | (fragrance content (wt %)) | | | | | | |
| Name of fragrant components | #1-A | #1-B | #1-C | #1-D | #1-E | #1-F | #1-G |
| Acetophenon | 47.44 | 47.44 | 47.44 | 47.44 | 47.44 | 47.44 | 47.44 |
| alpha-phenylethyl alcohol | 10.62 | 10.62 | 10.62 | 10.62 | 10.62 | 10.62 | 10.62 |
| Beta-phenylethyl alcohol | 4.78 | 4.78 | 4.78 | 4.78 | 4.78 | 4.78 | 4.78 |
| E-4,8-dimethyl-1,3,7- | 2.16 | 2.16 | 2.16 | 2.16 | 2.16 | 2.16 | 2.16 |
| nonatriene | | | | | | | |
| Linalool | 1.00 | 3.00 | 5.00 | 8.00 | 10.00 | 13.00 | 15.00 |
| 2-pentanone | 9.04 | 8.50 | 7.97 | 7.17 | 6.63 | 5.83 | 5.30 |
| 2-pentanol | 6.72 | 6.32 | 5.93 | 5.32 | 4.95 | 4.34 | 3.95 |
| Benzyl alcohol | 4.42 | 4.16 | 3.89 | 3.52 | 3.25 | 2.86 | 2.60 |
| cis-3-hexenyl acetate | 3.25 | 3.07 | 2.88 | 2.60 | 2.40 | 2.11 | 1.93 |
| Geraniol | 3.07 | 2.90 | 2.72 | 2.44 | 2.26 | 2.01 | 1.81 |
| 1-penten-3-ol | 2.90 | 2.73 | 2.56 | 2.30 | 2.13 | 1.88 | 1.71 |
| Acetoin | 2.33 | 2.19 | 2.05 | 1.85 | 1.71 | 1.50 | 1.37 |
| cis-3-hexenol | 1.55 | 1.46 | 1.37 | 1.23 | 1.14 | 1.00 | 0.91 |
| Linalool oxide | 0.58 | 0.55 | 0.51 | 0.46 | 0.43 | 0.38 | 0.34 |
| cis-4-hexenyl butyrate | 0.08 | 0.07 | 0.07 | 0.06 | 0.06 | 0.15 | 0.05 |
| Limonene | 0.06 | 0.05 | 0.05 | 0.05 | 0.04 | 0.04 | 0.03 |
| Total content | 100.00 | 100.00 | 100.00 | 100.00 | 100.00 | 100.00 | 100.00 |

1-7: Sensory Evaluation of Fragrance Compositions

The above perfume compositions #1-A, #1-B, #1-C, #1-D, #1-E, #1-F and #1-G were examined with respect to the similarity of fragrance to the fragrance of green tea flowers and the preference of fragrance using sensory evaluation in the same manner as described in Table 3 above. Meanwhile, after the comparative sensory evaluation of two perfume combinations was conducted, a breathing time of 5 minutes was given to eliminate olfactory paralysis. Sensory evaluation results for the similarity of fragrance between the new perfume compositions and green tea flowers, and the preference of fragrance, are shown in Table 8 below.

TABLE 8

| | Question 1 (similarity) | | | | | | | Question 2 (preference) | | | | | | |
|---|---|---|---|---|---|---|---|---|---|---|---|---|---|---|
| Evaluation | Perfume composition No. | | | | | | | Perfume composition No. | | | | | | |
| panels | #1-A | #1-B | #1-C | #1-D | #1-E | #1-F | #1-G | #1-A | #1-B | #1-C | #1-D | #1-E | #1-F | #1-G |
| 1 | 3.0 | 3.0 | 4.0 | 3.0 | 4.0 | 4.0 | 4.0 | 3.0 | 4.0 | 3.0 | 3.0 | 4.0 | 4.0 | 4.0 |
| 2 | 4.0 | 4.0 | 5.0 | 4.0 | 4.0 | 3.0 | 3.0 | 3.0 | 3.0 | 4.0 | 3.0 | 4.0 | 3.0 | 3.0 |
| 3 | 3.0 | 4.0 | 4.0 | 4.0 | 3.0 | 3.0 | 3.0 | 4.0 | 4.0 | 5.0 | 4.0 | 5.0 | 3.0 | 2.0 |
| 4 | 3.0 | 4.0 | 3.0 | 3.0 | 3.0 | 3.0 | 2.0 | 4.0 | 4.0 | 4.0 | 4.0 | 4.0 | 2.0 | 3.0 |
| 5 | 4.0 | 3.0 | 3.0 | 3.0 | 4.0 | 3.0 | 3.0 | 4.0 | 3.0 | 5.0 | 4.0 | 4.0 | 3.0 | 3.0 |
| 6 | 3.0 | 4.0 | 4.0 | 3.0 | 3.0 | 3.0 | 3.0 | 3.0 | 3.0 | 4.0 | 3.0 | 4.0 | 4.0 | 3.0 |
| 7 | 4.0 | 3.0 | 4.0 | 4.0 | 4.0 | 4.0 | 3.0 | 4.0 | 4.0 | 4.0 | 4.0 | 3.0 | 3.0 | 3.0 |
| 8 | 4.0 | 4.0 | 5.0 | 5.0 | 3.0 | 3.0 | 3.0 | 4.0 | 4.0 | 3.0 | 3.0 | 3.0 | 4.0 | 3.0 |
| 9 | 3.0 | 3.0 | 4.0 | 3.0 | 4.0 | 3.0 | 3.0 | 4.0 | 4.0 | 5.0 | 4.0 | 4.0 | 3.0 | 2.0 |
| 10 | 4.0 | 4.0 | 4.0 | 5.0 | 3.0 | 3.0 | 3.0 | 3.0 | 3.0 | 4.0 | 3.0 | 4.0 | 3.0 | 3.0 |
| 11 | 4.0 | 5.0 | 5.0 | 5.0 | 4.0 | 4.0 | 3.0 | 4.0 | 3.0 | 4.0 | 4.0 | 4.0 | 4.0 | 3.0 |
| 12 | 4.0 | 4.0 | 4.0 | 3.0 | 4.0 | 3.0 | 3.0 | 4.0 | 4.0 | 4.0 | 4.0 | 3.0 | 3.0 | 2.0 |
| 13 | 3.0 | 4.0 | 4.0 | 3.0 | 4.0 | 3.0 | 3.0 | 3.0 | 3.0 | 4.0 | 3.0 | 2.0 | 2.0 | 2.0 |
| 14 | 3.0 | 3.0 | 3.0 | 3.0 | 3.0 | 3.0 | 2.0 | 4.0 | 3.0 | 3.0 | 4.0 | 3.0 | 2.0 | 2.0 |
| 15 | 4.0 | 4.0 | 5.0 | 4.0 | 4.0 | 4.0 | 3.0 | 3.0 | 4.0 | 4.0 | 3.0 | 4.0 | 3.0 | 3.0 |
| 16 | 4.0 | 4.0 | 4.0 | 4.0 | 4.0 | 2.0 | 2.0 | 3.0 | 4.0 | 5.0 | 3.0 | 4.0 | 3.0 | 3.0 |
| 17 | 4.0 | 4.0 | 3.0 | 3.0 | 4.0 | 3.0 | 3.0 | 3.0 | 3.0 | 4.0 | 4.0 | 4.0 | 4.0 | 3.0 |
| 18 | 3.0 | 4.0 | 5.0 | 4.0 | 4.0 | 4.0 | 3.0 | 3.0 | 4.0 | 4.0 | 4.0 | 3.0 | 2.0 | 3.0 |
| 19 | 3.0 | 4.0 | 4.0 | 3.0 | 3.0 | 3.0 | 3.0 | 4.0 | 4.0 | 5.0 | 4.0 | 4.0 | 3.0 | 3.0 |
| 20 | 4.0 | 4.0 | 4.0 | 3.0 | 4.0 | 4.0 | 4.0 | 4.0 | 4.0 | 5.0 | 4.0 | 4.0 | 4.0 | 3.0 |
| Average | 3.55 | 3.80 | 4.05 | 3.60 | 3.65 | 3.25 | 2.95 | 3.55 | 3.60 | 4.15 | 3.60 | 3.70 | 3.10 | 2.80 |

As shown in Table 8, it could be seen that, when 1-10 wt % of E-4,8-dimethyl-1,3,7-nonatriene was added to the main fragrant components of green tea flowers, including acetophenon, alpha-phenylethyl alcohol, beta-phenylethyl alcohol and linalool, the resulting compositions could have a fragrance like that of green tea flower and show high preference (average of more than 3.5).

Meanwhile, it could be seen that, when the content of E-4,8-dimethyl-1,3,7-nonatriene was more than 10 wt %, the fragrance similarity and preference of the resulting perfume composition were decreased compared to those of perfume composition containing no E-4,8-dimethyl-1,3,7-nonatriene, rather than increased.

### Example 2

### Perfume Compositions Having Fragrance Like that of Green Tea Flowers, Based on Solvent Extraction Method

2-1: Analysis of Fragrance of Green Tea Flower Fragrance Using Solvent Extraction Method

To extract fragrant components from green tea flowers, 720 g of green tea flowers in bloom in Seogwang Dawon, Seogwang-ri, Jeju-do, Korea, were gathered, and divided into three 1-liter Erlenmeyer flasks. Then, 1.5 liters of ethyl ether (99.0% purity, Fisher Scientific) was poured into the flasks such that the flowers were completely submerged, and the flowers were immersed in the ether for 4 hours and 15 minutes. The contents in the flasks were filtered through Advantec No. 2 filter paper (185 mm diameter), and then the flowers were discarded, and the filtrate was dried over 100 g of anhydrous sodium sulfate (Na₂SO₄, 99.7% purity, Sigma Aldrich) to remove water. The residue was filtered again through Advantec No. 2 filter paper. Then, the solvent ethyl ether was volatilized in water bath at 38° C., and the residue was subjected to reduced-pressure steam distillation to yield 13.2 mg of a fragrance concentrate. The fragrance concentrate was placed in a non-contaminated glass bottle, which was then capped, sealed and stored in a refrigerator before use in analysis.

The fragrance concentrate thus obtained was placed at the inlet of GC-MS column and subjected to GC-MS analysis. The GC-MS analysis was carried out in the following manner.

### Analysis Conditions

Analysis instrument: HP 6890 II GC

### Detector: HP 5973 MSD

Column: PEG-20M (60 m×0.25 mm×0.25 um)

### Carrier gas: He

Injection temperature: 250° C.
Detection temperature: 280° C.
Oven temperature: 70-220° C. (3° C./min)
Ion voltage: 70 eV
Injection volume: 1 μl
Split ratio: 1:40

As a result, the fragrant components of green tea flowers, analyzed by the solvent extraction method, are shown in Table 9 below.

TABLE 9

| Name of fragrant components | Content (wt %) |
|---|---|
| Acetophenon | 31.10 |
| alpha-methylbenzyl alcohol | 23.60 |
| 2-phenylethyl alcohol | 10.15 |
| Linalool | 3.53 |
| Geranoil | 1.94 |
| Linalool oxide | 2.25 |
| Methyl salicylate | 0.11 |
| Indole | 0.05 |
| Vanilin | 0.04 |
| Others | 27.23 |
| Total content | 100.00 |

As shown in Table 9, green tea flowers contained, as main fragrant components, acetophenon, alpha-methylbenzyl alcohol, and 2-phenylethyl alcohol, and the content of these main components in the total fragrant components was 64.85%.

2-2. Comparative Sensory Evaluation of Fragrance Between Perfume Composition Based on Analysis Results and Natural Green Tea Flowers

Based on the above analysis results, a new perfume composition (sample C) shown in Table 10 below was prepared, and the similarity of fragrance between the perfume composition (sample C) and natural green tea flowers was examined through sensory evaluation.

TABLE 10

| Name of fragrant components | Content (wt) |
|---|---|
| Acetophenon | 31.10 |
| alpha-methylbenzyl alcohol | 23.60 |
| 2-phenylethyl alcohol | 10.15 |
| Linalool | 3.53 |
| Geranoil | 1.94 |
| Linalool oxide | 2.25 |
| Methyl salicylate | 0.11 |
| Indole | 0.05 |
| Vanillin | 0.04 |
| DPG (Solvent) | 27.23 |
| Total content | 100.00 |

The sensory evaluation was conducted on twenty 20-45-year-old general men and women, and the similarity of fragrance (question 1) between the green tea flowers in full bloom (sample A) and the perfume composition (sample C) obtained using the solvent extraction method, and the preference of fragrance (question 2), were examined by causing the evaluation panels to smelling the fragrance of each of the samples A and C and answering the questionnaire shown in Table 3 above. The evaluation results are shown in Table 11 below.

TABLE 11

| Evaluation | Question 1 | Question 2 |
|---|---|---|
| panel No. | (similarity) | (preference) |
| 1 | 5 | 5 |
| 2 | 2 | 5 |
| 3 | 3 | 2 |
| 4 | 2 | 5 |
| 5 | 5 | 2 |
| 6 | 3 | 5 |
| 7 | 2 | 5 |
| 8 | 5 | 4 |
| 9 | 2 | 2 |
| 10 | 2 | 2 |
| 11 | 5 | 5 |
| 12 | 5 | 3 |
| 13 | 3 | 2 |
| 14 | 5 | 5 |
| 15 | 3 | 3 |
| 16 | 4 | 2 |
| 17 | 3 | 2 |
| 18 | 2 | 5 |
| 19 | 5 | 2 |
| 20 | 4 | 5 |
| Average | 3.5 | 3.55 |

As shown in Table 11, the fragrance and preference of the perfume composition (sample C) were somewhat similar to those of natural green tea flowers, but were not satisfactory.

2-3: Analysis of Fragrance and Comparison of Fragrance with Green Tea Flowers, Conducted by Professional Evaluation Group

As described above, the perfume composition consisting of the components analyzed by the solvent extraction method had low similarity to the fragrance of green tea flowers. Thus, each of the fragrant components of green tea flowers was subjected to sensory evaluation by a professional evaluation group consisting of flavorists. As a result, it was found that, among the fragrant components of green tea flowers, three components, including acetophenon emitting orange flower fragrance, alpha-methylbenzyl alcohol emitting Gardenia fragrance, and 2-phenylethyl alcohol emitting rose fragrance, were main components making the characteristic fragrance of green tea flowers.

Meanwhile, it was confirmed through sensory evaluation that fragrant components, including linalool, geraniol, linalool oxide, methyl salicylate, indole and vanillin, classified as the main fragrant components together with acetophenon, alpha-methylbenzyl alcohol and 2-phenylethyl alcohol in the solvent extraction analysis results, had no particular effect on making the fragrance of green tea flowers.

2-4: Preparation of New Perfume Compositions Having Varying Contents of Three Main Components

From the results of Example 2-3, it could be seen that the main components for the characteristic fragrance of green tea flowers were acetophenon, alpha-methylbenzyl alcohol and 2-phenylethyl alcohol. Thus, in order to prepare a perfume composition, which contains these three components to emit the fragrance of green tea flowers and, at the same time, has high preference, the present inventors made new perfume compositions #2-1 to #2-6. The perfume compositions #2-1 to #2-6 were prepared such that the ratio between these three components was maintained constant while the contents of the three components in the compositions shown in Table 12 below were changed in the range of 50-75 wt %.

TABLE 12

| Name | | | | | | |
|---|---|---|---|---|---|---|
| of fragrant | #2-1 | #2-2 | #2-3 | #2-4 | #2-5 | #2-6 |
| components | (50%) | (55%) | (60%) | (65%) | (70%) | (75%) |
| Acetophenon | 23.98 | 26.38 | 28.77 | 31.17 | 33.57 | 35.97 |
| alpha- | 18.20 | 20.02 | 21.84 | 23.65 | 25.47 | 27.29 |
| methylbenzyl | | | | | | |
| alcohol | | | | | | |
| 2-phenylethyl | 7.83 | 8.61 | 9.39 | 10.17 | 10.96 | 11.74 |
| alcohol | | | | | | |
| Linalool | 3.53 | 3.53 | 3.53 | 3.53 | 3.53 | 3.53 |
| Geranoil | 1.94 | 1.94 | 1.94 | 1.94 | 1.94 | 1.94 |
| Linalool oxide | 2.25 | 2.25 | 2.25 | 2.25 | 2.25 | 2.25 |
| Methyl | 0.11 | 0.11 | 0.11 | 0.11 | 0.11 | 0.11 |
| salicylate | | | | | | |
| Indole | 0.05 | 0.05 | 0.05 | 0.05 | 0.05 | 0.05 |
| Vanillin | 0.04 | 0.04 | 0.04 | 0.04 | 0.04 | 0.04 |
| DPG (Solvent) | 42.08 | 37.08 | 32.08 | 27.08 | 22.08 | 17.08 |
| Total content | 100.0 | 100.0 | 100.0 | 100.0 | 100.0 | 100.0 |

2-5: Sensory Evaluation of Perfume Compositions

Among the six new perfume compositions, the five perfume compositions except for the perfume composition #2-4 having components and contents similar to those of the perfume composition evaluated in Example 2-2 were examined with respect to the similarity of fragrance to the fragrance of green tea flowers and the preference of fragrance thereof using sensory evaluation in the same manner as described in Table 3 above. Meanwhile, after the comparative sensory evaluation of two perfume combinations was conducted, a breathing time of 5 minutes was given to eliminate olfactory paralysis. Sensory evaluation results for the similarity of fragrance between the new perfume compositions and green tea flowers, and the preference of fragrance, are shown in Table 13 below.

TABLE 13

| | Question 1 (similarity) | | | | | Question 2 (preference) | | | | |
|---|---|---|---|---|---|---|---|---|---|---|
| | Perfume composition No. | | | | | Perfume composition No. | | | | |
| Evalution panels | #2-1 | #2-2 | #2-3 | #2-5 | #2-6 | #2-1 | #2-2 | #2-3 | #2-5 | #2-6 |
| 1 | 2 | 4 | 3 | 5 | 3 | 2 | 3 | 5 | 5 | 1 |
| 2 | 2 | 3 | 3 | 3 | 2 | 3 | 3 | 3 | 5 | 1 |
| 3 | 3 | 3 | 4 | 3 | 1 | 3 | 4 | 3 | 5 | 2 |
| 4 | 2 | 3 | 3 | 2 | 2 | 4 | 3 | 5 | 3 | 2 |
| 5 | 2 | 4 | 2 | 2 | 1 | 3 | 3 | 2 | 4 | 2 |
| 6 | 2 | 4 | 3 | 5 | 1 | 3 | 4 | 3 | 3 | 2 |
| 7 | 3 | 4 | 2 | 2 | 2 | 2 | 5 | 3 | 3 | 2 |
| 8 | 2 | 3 | 3 | 5 | 2 | 3 | 5 | 2 | 3 | 4 |
| 9 | 3 | 4 | 5 | 2 | 2 | 2 | 3 | 2 | 3 | 2 |
| 10 | 2 | 4 | 5 | 5 | 2 | 3 | 4 | 2 | 5 | 2 |
| 11 | 2 | 3 | 5 | 5 | 2 | 3 | 3 | 3 | 5 | 2 |
| 12 | 3 | 4 | 5 | 3 | 3 | 2 | 3 | 5 | 5 | 3 |
| 13 | 2 | 2 | 2 | 5 | 3 | 2 | 3 | 5 | 2 | 2 |
| 14 | 3 | 2 | 3 | 5 | 2 | 3 | 2 | 3 | 2 | 1 |
| 15 | 3 | 4 | 3 | 5 | 1 | 2 | 3 | 5 | 2 | 3 |
| 16 | 2 | 3 | 3 | 5 | 1 | 3 | 5 | 5 | 2 | 2 |
| 17 | 2 | 3 | 5 | 2 | 3 | 4 | 5 | 5 | 3 | 2 |
| 18 | 1 | 4 | 5 | 3 | 2 | 2 | 4 | 3 | 5 | 1 |
| 19 | 2 | 4 | 5 | 5 | 3 | 1 | 4 | 4 | 5 | 2 |
| 20 | 2 | 5 | 3 | 5 | 1 | 1 | 3 | 5 | 5 | 2 |
| Average | 2.25 | 3.5 | 3.6 | 3.85 | 1.95 | 2.55 | 3.6 | 3.65 | 3.75 | 2 |

As shown in Table 13, the sample #2-5, in which the content of the three main components was 70 wt %, showed the highest similarity and preference, but all the similarity and preference of the sample #2-5 were not as high as expected.

1-6: Preparation of Perfume Compositions Having Improved Fragrance, Based on Analysis Conducted by Professional Evaluation Group

To improve the similarity and preference of the sample #2-5, which was shown to be most similar to the fragrance of green tea flowers in Table 13 above, new perfume compositions were prepared by adding lilac alcohol emitting elegant fragrance to the sample #2-5 in which the total content of acetophenon, alpha-methylbenzyl alcohol and 2-phenylethyl alcohol was 70%. The lilac alcohol was added at concentrations of 0.1%, 0.5%, 1.0%, 2.5%, 5.0%, 7.5% and 10%. These perfume compositions are shown in Table 14 below.

TABLE 14

| | Perfume composition No. | | | | | | |
|---|---|---|---|---|---|---|---|
| | #2-A | #2-B | #2-C | #2-D | #2-E | #2-F | #2-G |
| Name of fragrant | Content | Content | Content | Content | Content | Content | Content |
| components | (%) | (%) | (%) | (%) | (%) | (%) | (%) |
| Acetophenon | 33.57 | 33.57 | 33.57 | 33.57 | 33.57 | 33.57 | 33.57 |
| alpha-methylbenzyl | 25.47 | 25.47 | 25.47 | 25.47 | 25.47 | 25.47 | 25.47 |
| alcohol | | | | | | | |
| 2-phenylethyl alcohol | 10.96 | 10.96 | 10.96 | 10.96 | 10.96 | 10.96 | 10.96 |
| Linalool | 3.53 | 3.53 | 3.53 | 3.53 | 3.53 | 3.53 | 3.53 |
| Geranoil | 1.94 | 1.94 | 1.94 | 1.94 | 1.94 | 1.94 | 1.94 |
| Linalool oxide | 2.25 | 2.25 | 2.25 | 2.25 | 2.25 | 2.25 | 2.25 |
| Methyl salicylate | 0.11 | 0.11 | 0.11 | 0.11 | 0.11 | 0.11 | 0.11 |
| Indole | 0.05 | 0.05 | 0.05 | 0.05 | 0.05 | 0.05 | 0.05 |
| Vanillin | 0.04 | 0.04 | 0.04 | 0.04 | 0.04 | 0.04 | 0.04 |
| Lilac alcohol | 0.1 | 0.5 | 1.0 | 2.5 | 5.0 | 7.5 | 10.0 |
| DPG (Solvent) | 21.98 | 21.58 | 21.08 | 19.58 | 17.07 | 14.58 | 12.08 |
| Total content | 100.00 | 100.00 | 100.00 | 100.00 | 100.00 | 100.00 | 100.00 |

2-7: Sensory Evaluation of Fragrance Compositions

The above perfume compositions #2-A, #2-B, #2-C, #2-D, #2-E, #2-F and #2-G were examined with respect to the similarity of fragrance to green tea flowers and the preference of fragrance using sensory evaluation in the same manner as described in Table 3 above. Meanwhile, after the comparative sensory evaluation of two perfume combinations was conducted, a breathing time of 5 minutes was given to eliminate olfactory paralysis. Sensory evaluation results for the similarity of fragrance between the new perfume compositions and green tea flowers, and the preference of fragrance, are shown in Table 15 below.

TABLE 15

| | Question 1 (similarity) | | | | | | | Question 2 (preference) | | | | | | |
|---|---|---|---|---|---|---|---|---|---|---|---|---|---|---|
| Evaluation | Perfume composition No. | | | | | | | Perfume composition No. | | | | | | |
| panels | #2-A | #2-B | #2-C | #2-D | #2-E | #2-F | #2-G | #2-A | #2-B | #2-C | #2-D | #2-E | #2-F | #2-G |
| 1 | 5 | 5 | 4 | 5 | 4 | 4 | 4 | 5 | 5 | 3 | 5 | 5 | 4 | 4 |
| 2 | 4 | 5 | 5 | 4 | 4 | 3 | 3 | 5 | 5 | 4 | 5 | 5 | 3 | 3 |
| 3 | 5 | 5 | 4 | 5 | 5 | 3 | 3 | 4 | 4 | 5 | 4 | 5 | 3 | 2 |
| 4 | 3 | 4 | 3 | 3 | 5 | 3 | 2 | 4 | 4 | 4 | 4 | 4 | 2 | 3 |
| 5 | 4 | 5 | 3 | 3 | 4 | 3 | 3 | 4 | 5 | 5 | 4 | 4 | 3 | 3 |
| 6 | 3 | 4 | 4 | 5 | 5 | 3 | 3 | 5 | 5 | 4 | 3 | 4 | 4 | 3 |
| 7 | 4 | 5 | 4 | 4 | 4 | 4 | 3 | 4 | 4 | 4 | 4 | 5 | 3 | 3 |
| 8 | 4 | 5 | 5 | 5 | 3 | 3 | 3 | 4 | 5 | 3 | 5 | 5 | 4 | 3 |
| 9 | 5 | 5 | 4 | 5 | 4 | 3 | 3 | 4 | 5 | 5 | 4 | 4 | 3 | 2 |
| 10 | 5 | 5 | 4 | 5 | 3 | 3 | 3 | 3 | 5 | 4 | 3 | 4 | 3 | 3 |
| 11 | 4 | 5 | 5 | 5 | 5 | 4 | 3 | 4 | 5 | 4 | 4 | 4 | 4 | 3 |
| 12 | 5 | 5 | 4 | 3 | 5 | 3 | 3 | 4 | 4 | 4 | 4 | 3 | 3 | 2 |
| 13 | 5 | 5 | 4 | 3 | 5 | 3 | 3 | 3 | 5 | 4 | 5 | 2 | 2 | 2 |
| 14 | 5 | 5 | 3 | 3 | 3 | 3 | 2 | 4 | 5 | 3 | 4 | 3 | 2 | 2 |
| 15 | 5 | 4 | 5 | 4 | 4 | 4 | 3 | 5 | 4 | 4 | 3 | 4 | 3 | 3 |
| 16 | 4 | 4 | 4 | 4 | 4 | 2 | 2 | 5 | 5 | 5 | 5 | 5 | 3 | 3 |
| 17 | 4 | 4 | 3 | 5 | 4 | 3 | 3 | 5 | 5 | 4 | 4 | 5 | 4 | 3 |
| 18 | 3 | 4 | 5 | 4 | 4 | 4 | 3 | 3 | 5 | 4 | 4 | 5 | 2 | 3 |
| 19 | 5 | 5 | 4 | 5 | 3 | 3 | 3 | 4 | 5 | 5 | 4 | 4 | 3 | 3 |
| 20 | 4 | 5 | 4 | 3 | 4 | 4 | 4 | 4 | 5 | 5 | 4 | 4 | 4 | 3 |
| Average | 4.3 | 4.7 | 4.05 | 4.15 | 4.1 | 3.25 | 2.95 | 4.15 | 4.75 | 4.15 | 4.1 | 4.2 | 3.1 | 2.8 |

As shown in Table 15, it could be seen that, when 0.1-5.0 wt % of lilac alcohol was added to the main fragrant components of green tea flowers, including acetophenon, alpha-methylbenzyl alcohol and 2-phenylethyl alcohol, the resulting compositions could have a fragrance like that of green tea flowers and show high preference.

Meanwhile, it could be seen that, when the content of lilac alcohol was more than 5.0 wt %, the fragrance similarity and preference of the resulting perfume composition were decreased compared to those of perfume composition containing no lilac alcohol, rather than increased.

### Preparation Example

A perfume shown in Table 16 below was prepared using the above-described perfume composition having similarity to the fragrance of green tea flowers.

TABLE 16
Perfume containing perfume composition having
fragrance like that of green tea flowers

| | |
|---|---|
| Perfume composition having fragrance | 15 wt % |
| like that of green tea flowers | |
| Alcohol | 83 wt % |
| Water | 2 wt % |
| Pigment | q.s. |
| UV-blocking agent | q.s. |

### INDUSTRIAL APPLICABILITY

As described above, the perfume composition according to the present invention contains trans-4,8-dimethyl-1,3,7-nonatriene or lilac alcohol, in addition to main fragrant components of green tea flowers, including acetophenon and phenylethyl alcohol, analyzed by the SPME method or the solvent extraction method. Thus, the inventive perfume composition can emit a fragrance like the characteristic fragrance of green tea flowers, and can also have high preference of fragrance.

1. A method for preparing a perfume composition having a fragrance like that of green tea flowers, the method comprising the steps of:
analyzing fragrant components of green tea flowers;
selecting, from among the analyzed fragrant components, main fragrant components of green tea flowers, having high similarity to the fragrance of green tea flowers and high preference of fragrance; and
adding a fragrance-enhancing perfume to the main fragrant components.

2. The method of claim 1, wherein an SPME method is used to analyze the fragrant components of green tea flowers.

3. The method of claim 1, wherein a solvent extraction method is used to analyze the fragrant components of green tea flowers.

4. A perfume composition having a fragrance like that of green tea flowers, which is prepared according to the method of claim 1.

5. A perfume composition having a fragrance like that of green tea flowers, which is prepared according to the method of claim 1.

6. The perfume composition of claim 4, which contains, as active ingredients, acetophenon, alpha-phenylethyl alcohol, beta-phenylethyl alcohol, linalool and E-4,8-dimethyl-1,3,7-nonatriene.

7. The perfume composition of claim 6, which additionally contains 2-pentanone, 2-pentanol, benzyl alcohol, Z-3-hexenyl acetate, geraniol, 1-penten-3-ol, acetoin, Z-3-hexenol, linalool oxide, Z-4-hexenyl butyrate and limonene.

8. The perfume composition of claim 6, which contains, based on the total weight of the composition, 44.00-51.00 wt % of acetophenon, 8.20-13.00 wt % of alpha-phenylethyl alcohol, 3.50-6.20 wt % of beta-phenylethyl alcohol, 1.05-3.50 wt % of linalool and 1-10 wt % of E-4,8-dimethyl-1,3,7-nonatriene.

9. The perfume composition of claim 7, which additionally contains, based on the total weight of the composition, 6.00-9.97 wt % of 2-pentanone, 4.20-7.23 wt % of 2-pentanol, 2.75-4.95 wt % of benzyl alcohol, 2.00-3.75 wt % of Z-3-hexenyl acetate, 1.76-3.57 wt % of geraniol, 2.36-2.40 wt % of 1-penten-3-ol, 1.21-2.83 wt % of acetoin, 0.64-1.05 wt % of Z-3-hexenol, 0.23-0.78 wt % of linalool oxide, 0.02-0.10 wt % of Z-4-hexenyl butyrate and 0.01-0.07 wt % of limonene.

10. The perfume composition of claim 5, which contains, as active ingredients, acetophenon, alpha-methylbenzyl alcohol, 2-phenylethyl alcohol and lilac alcohol.

11. The perfume composition of claim 10, which additionally contains linalool, geraniol, linalool oxide, methyl salicylate, indole and vanillin.

12. The perfume composition of claim 10, which contains, based on the total weight of the composition, 26.0-35.0 wt % of acetophenon, 20.0-27.0 wt % of alpha-methylbenzyl alcohol, 8.5-11.0 wt % of 2-phenylethyl alcohol and 0.1-7.4 wt % of lilac alcohol.

13. The perfume composition of claim 11, which additionally contains, based on the total weight of the composition, 3.0-4.0 wt % of linalool, 1.5-2.5 wt % of geraniol, 2.0-3.0 wt % of linalool oxide, 0.1-1.0 wt % of methyl salicylate, 0.01-0.1 wt % of indole and 0.01-0.1 wt % of vanillin.

14. A cosmetic composition containing the perfume composition of claim 4 in an amount of 0.1-25.0 wt % based on the total weight of the cosmetic composition.
